(19) United States
(12) Patent Application Publication
(10) Pub. No.: US 2026/0363758 A1
(43) Pub. Date: Nov. 19, 2026

(54) CAM SLOT CONFIGURATIONS FOR SURGICAL INSTRUMENTS

(71) Applicant: Covidien LP, Mansfield, MA (US)

(72) Inventor: Monte S. FRY, Longmont, CO (US)

(21) Appl. No.: 19/737,228

(22) Filed: Jul. 10, 2026

Related U.S. Application Data
(63) Continuation of application No. PCT/IB2025/050235, filed on Jan. 9, 2025.
(60) Provisional application No. 63/620,541, filed on Jan. 12, 2024.

(51) Int. Cl. A61B 18/14; A61B 17/29

(52) U.S. Cl. CPC A61B 18/1447; A61B 2017/2934; A61B 2017/2936

(57) ABSTRACT

A surgical instrument includes a pivot pin, a cam pin, and first and second jaw members pivotably coupled to one another about the pivot pin. The first jaw member defines a cam slot having the cam pin received therein such that movement of the cam pin through the cam slot pivots the first jaw member relative to the second jaw member from a spaced apart position towards an approximated position to grasp tissue between the first and second jaw members. At least a portion of the cam slot defines an elliptical arc to facilitate application of a substantially constant jaw force from the first jaw member to tissue grasped between the first and second jaw members when the cam pin is disposed at any position within the portion of the cam slot.

### CROSS REFERENCE TO RELATED APPLICATIONS

This application is a continuation of International Patent Application No. PCT/IB2025/050235, filed Jan. 9, 2025, which claims the benefit of U.S. Provisional Patent Application Ser. No. 63/620,541, filed Jan. 12, 2024, which applications are incorporated herein by reference. To the extent appropriate, a claim of priority is made to each of the above disclosed applications.

### FIELD

The present disclosure relates to surgical instruments and, more particularly, to cam slot configurations for surgical instruments such as, for example, to provide substantially constant jaw force to tissue grasped between jaw members of the surgical instrument.

### BACKGROUND

Various different surgical instruments employ cam mechanisms to actuate one or more operable components of the surgical instrument. A surgical forceps, for example, is a pliers-like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Such electrosurgical forceps may include one or more cam mechanisms to enable selective closure of the jaw members to grasp tissue for treating the grasped tissue.

### SUMMARY

As used herein, the term “distal” refers to the portion that is being described which is farther from an operator (whether a human user or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Further, to the extent consistent, any or all of the aspects detailed herein may be used in conjunction with any or all of the other aspects detailed herein.

Provided in accordance with the present disclosure is a surgical instrument including a pivot pin, a cam pin, and first and second jaw members pivotably coupled to one another about the pivot pin. The first jaw member defines a cam slot having the cam pin received therein such that movement of the cam pin through the cam slot pivots the first jaw member relative to the second jaw member from a spaced apart position towards an approximated position to grasp tissue between the first and second jaw members. The second jaw member may be fixed (defining a unilateral configuration) or may also pivot relative to the first jaw member between the spaced apart and approximated positions (defining a bilateral configuration). At least a portion of the cam slot defines an elliptical arc to facilitate application of a substantially constant jaw force from the first jaw member to tissue grasped between the first and second jaw members when the cam pin is disposed at any position within the portion of the cam slot.

In an aspect of the present disclosure, the elliptical arc defines an eccentricity of from about 0.70 to about 0.90.

In another aspect of the present disclosure, the elliptical arc deviates from a perfect ellipse by less than or equal to about 0.006 inches at any point along the elliptical arc in either direction normal to the perfect ellipse.

In still another aspect of the present disclosure, the elliptical arc is disposed between a vertex and a co-vertex of an ellipse defining the elliptical arc. In another aspect, the elliptical arc intersects a vertex or co-vertex of an ellipse defining the elliptical arc.

In yet another aspect of the present disclosure, the portion of the cam slot is an operative portion of the cam slot defined between a proximal-most position of the cam pin within the cam slot and a distal-most position of the cam pin within the cam slot. In such aspects, the proximal-most position may be distally spaced from a proximal end of the cam slot and/or the distal-most position may be proximally spaced from a distal end of the cam slot. As such, the portion of the cam slot may be less than an entire extent of the cam slot.

In still yet another aspect of the present disclosure, the surgical instrument further includes a drive assembly including a compression spring and a drive shaft coupling the compression spring with the cam pin such that translation of the compression spring translates the drive shaft to thereby move the cam pin through the cam slot. In such aspects, the cam slot may define a first section and a second section. Movement of the cam pin through the first section of the cam slot is substantially independent of a spring force associated with the compression spring and wherein movement of the cam pin through the second section of the cam slot is dependent upon the spring force associated with the compression spring. The portion of the cam slot may include the first section and/or the second section.

In another aspect of the present disclosure, the cam slot is configured such that movement of the cam pin a distance through a first section of the cam slot pivots the first jaw member relative to the second jaw member a first angular amount and wherein movement of the cam pin the distance through a second section of the cam slot pivots the first jaw member relative to the second jaw member a second, different angular amount.

Another surgical instrument provided in accordance with the present disclosure includes a drive assembly, a cam pin, and first and second jaw members. The drive assembly includes a compression spring and a drive shaft. Translation of the compression spring translates the drive shaft. The drive shaft is engaged to the cam pin such that translation of the drive shaft moves the cam pin. The first and second jaw members are pivotably coupled to one another and the first jaw member defines a cam slot having the cam pin received therein such that movement of the cam pin through the cam slot pivots the first jaw member relative to the second jaw member from a spaced apart position towards an approximated position to grasp tissue between the first and second jaw members. The second jaw member may be fixed (defining a unilateral configuration) or may also pivot relative to the first jaw member between the spaced apart and approximated positions (defining a bilateral configuration). At least a portion of the cam slot defines an elliptical arc to facilitate application of a substantially constant jaw force from the first jaw member to tissue grasped between the first and second jaw members when the cam pin is disposed at any position within the portion of the cam slot.

In an aspect of the present disclosure, the elliptical arc defines an eccentricity of from about 0.70 to about 0.90.

In another aspect of the present disclosure, the elliptical arc deviates from a perfect ellipse by less than or equal to about 0.006 inches at any point along the elliptical arc in either direction normal to the perfect ellipse.

In still another aspect of the present disclosure, the elliptical arc is disposed between a vertex and a co-vertex of an ellipse defining the elliptical arc. In another aspect, the elliptical arc intersects a vertex or co-vertex of an ellipse defining the elliptical arc.

In yet another aspect of the present disclosure, the portion of the cam slot is an operative portion of the cam slot defined between a proximal-most position of the cam pin within the cam slot and a distal-most position of the cam pin within the cam slot. In such aspects, the proximal-most position may be distally spaced from a proximal end of the cam slot and/or the distal-most position may be proximally spaced from a distal end of the cam slot. As such, the portion of the cam slot may be less than an entire extent of the cam slot.

In still yet another aspect of the present disclosure, the cam slot defines a first section and a second section. Movement of the cam pin through the first section of the cam slot is substantially independent of a spring force associated with the compression spring and wherein movement of the cam pin through the second section of the cam slot is dependent upon the spring force associated with the compression spring. The portion of the cam slot may include the first section and/or the second section.

In another aspect of the present disclosure, the cam slot is configured such that movement of the cam pin a distance through a first section of the cam slot pivots the first jaw member relative to the second jaw member a first angular amount and wherein movement of the cam pin the distance through a second section of the cam slot pivots the first jaw member relative to the second jaw member a second, different angular amount.

In another aspect of the present disclosure, the drive assembly is driven by a manually-actuated handle. Alternatively, the drive assembly may be driven by a robotic arm.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a side view of an electrosurgical system in accordance with the present disclosure including an electrosurgical forceps and an electrosurgical generator;

FIG. 2 is a side, cutaway view of a proximal portion of the electrosurgical forceps of the system of FIG. 1;

FIG. 3 is a top, longitudinal, cross-sectional view taken along section line 3-3 of FIG. 2;

FIG. 4 is a perspective view of an end effector assembly of the electrosurgical forceps with first and second jaw members of the end effector assembly disposed in a spaced apart position;

FIG. 5 is a side view of the end effector assembly of the electrosurgical forceps with the first and second jaw members disposed in a first approximated position;

FIG. 6 is a side view of the end effector assembly of the electrosurgical forceps with the first and second jaw members disposed in a second, further approximated position;

FIG. 7 is a side view of a proximal portion of one of the jaw members of the end effector assembly of the electrosurgical forceps including a cam slot configuration in accordance with the present disclosure;

FIG. 8 is a side view of another jaw member configured for use with the end effector assembly of the electrosurgical forceps and including another cam slot configuration in accordance with the present disclosure;

FIG. 9 is a schematic illustration of a robotic surgical system provided in accordance with the present disclosure; and

FIG. 10 is a perspective view of another electrosurgical forceps provided in accordance with the present disclosure and configured for use with the robotic surgical system of FIG. 9.

### DETAILED DESCRIPTION

The present disclosure provides cam slot configurations for surgical instruments such as, for example, electrosurgical forceps. The cam slot configurations of the present disclosure provide substantially constant jaw force to tissue grasped between jaw members of the electrosurgical forceps through a wide range of jaw angles (defined as the angle between the tissue contacting surfaces of the jaw members), thus enabling appropriate application of jaw force to tissues of various thicknesses and/or compressibilities to facilitate treating, e.g., sealing, tissues of different thicknesses and/or compressibilities including, in aspects, vessels up to and, in aspects, greater than, 10 mm in diameter. However, the aspects and features of the present disclosure are also applicable to other surgical instruments and/or for other purposes.

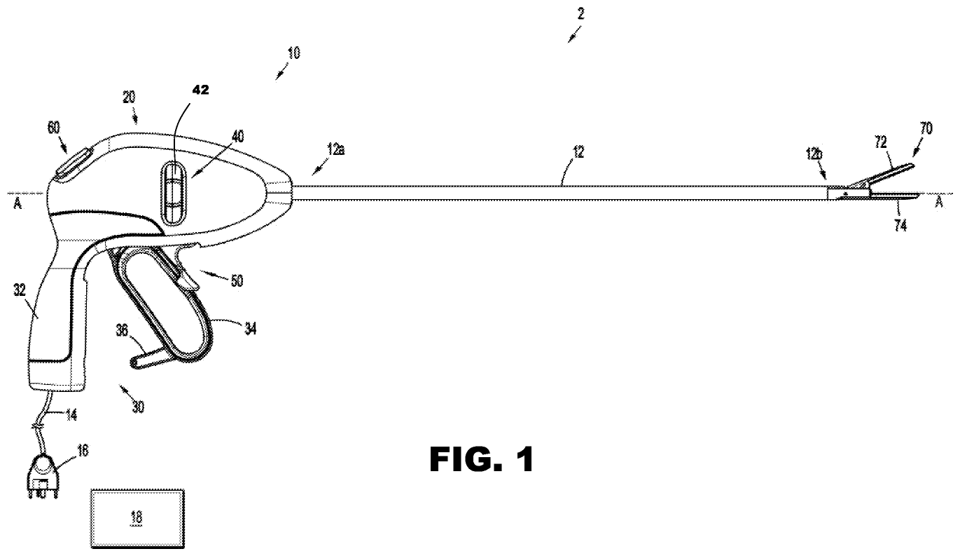

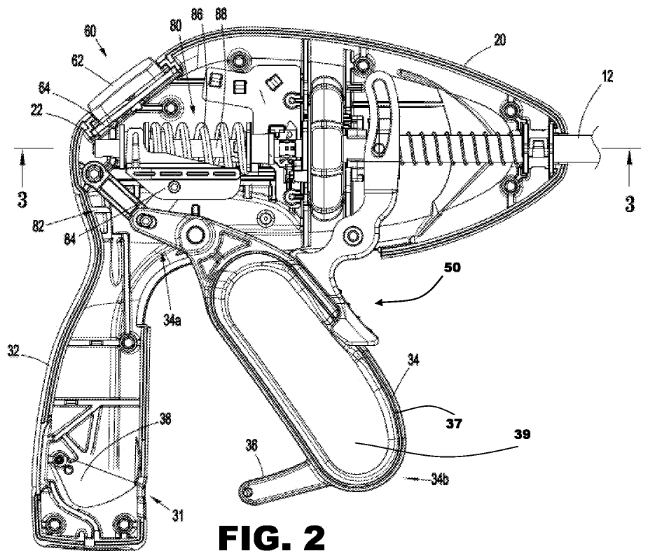

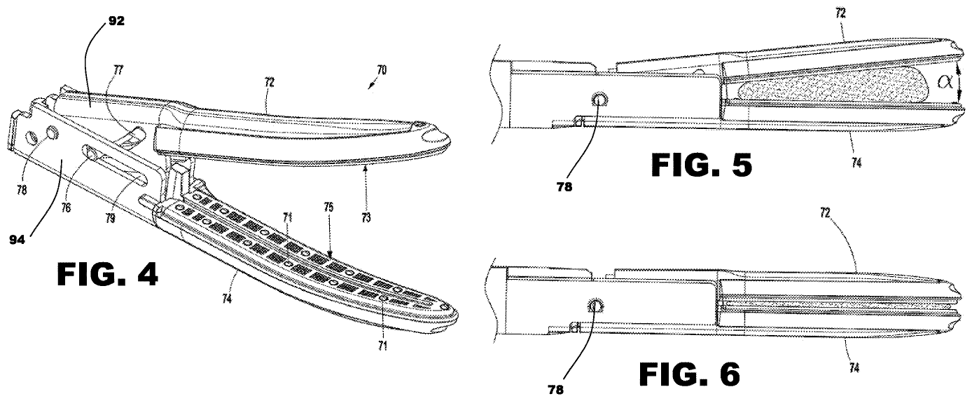

Referring to FIG. 1, an electrosurgical system 2 provided in accordance with the present disclosure includes an electrosurgical forceps 10 and an electrosurgical generator 18. Forceps 10 includes a housing 20, a handle assembly 30, a rotating assembly 40, a trigger assembly 50, an activation assembly 60, an end effector assembly 70, and a drive assembly 80 (FIG. 2). End effector assembly 70 includes first and second jaw members 72, 74, at least one of which is pivotable relative to the other about a pivot pin 78 (FIG. 4) to grasp tissue. Reference to a pivot pin, as utilized herein, includes both continuous pins as well as split pins, e.g., wherein first and second pin portions cooperate to define the pivot pin. End effector assembly 70 may further include a knife (not shown) operably coupled to trigger assembly 50 to enable selective translation of the knife between jaw members 72, 74 to cut tissue (e.g., previously sealed tissue) grasped between jaw members 72, 74. Alternatively, end effector assembly 70 may include an energizable cutting element, e.g., disposed on either or both jaw members 72, 74, configured to cut tissue grasped between jaw members 72, 74 upon energization of the energizable cutting element.

Forceps 10 further includes an outer shaft 12 defining a longitudinal axis “A-A.” Outer shaft 12 has a proximal end portion 12a operatively engaged to housing 20 and a distal end portion 12b operably engaged to end effector assembly 70. Forceps 10 also includes an electrosurgical cable 14 having a plug 16 configured to connect forceps 10 to generator 18, e.g., to enable generator 18 to communicate with forceps 10 and control the supply of electrosurgical energy to end effector assembly 70 of forceps 10 for sealing tissue grasped between first and second jaw members 72, 74.

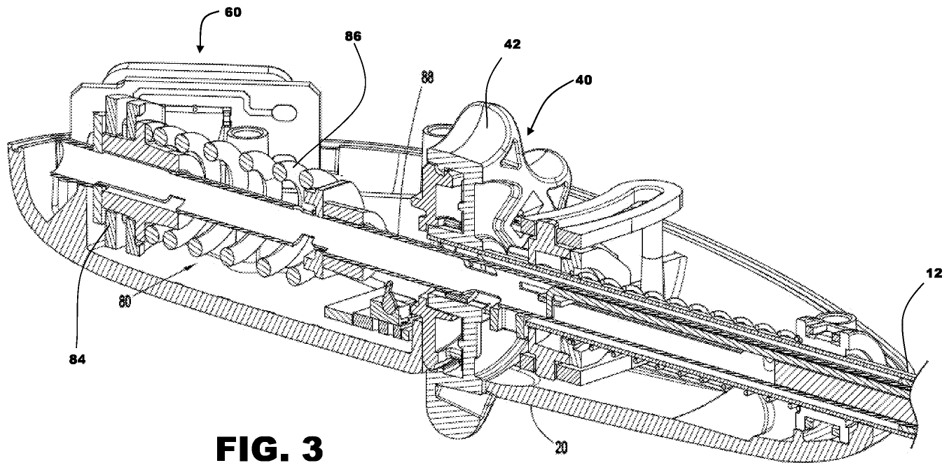

With additional reference to FIGS. 2 and 3, handle assembly 30 includes a fixed handle 32 and a movable handle 34. Fixed handle 32 is integrally associated with housing 20 and movable handle 34 is movable relative to fixed handle 32 to actuate drive assembly 80 of forceps 10. More specifically, movable handle 34 has an upper end portion 34a that is pivotally secured within housing 20 and operably engaged with drive assembly 80. Drive assembly 80 includes a plurality of linkages 82, a carriage 84, a compression spring 86, and a drive shaft 88. Upper end portion 34a of movable handle 34 operably couples to linkages 82 that cooperate to move carriage 84 distally against compression spring 86 in response to actuation of movable handle 34 towards fixed handle 32. Movement of carriage 84 distally against compression spring 86, in turn, regulates translation of drive shaft 88 through outer shaft 12 and relative to end effector assembly 70 to move either or both of jaw members 72, 74 relative to one another to grasp tissue between jaw members 72, 74 and regulate the closure force applied to tissue grasped between jaw members 72, 74.

Referring also to FIG. 4, movement of carriage 84 distally against compression spring 86, more specifically, initially urges compression spring 86 to translate distally to thereby translate drive shaft 88 distally through outer shaft 12 to drive relative movement of a cam pin 76 (that is secured relative to a distal end portion of drive shaft 88) through cam slots 77, 79 defined within respective first and second jaw members 72, 74 to thereby urge jaw member 72 to pivot about pivot pin 78 and relative to jaw member 74 from a spaced apart position (FIG. 4) towards an appropriate approximated position (FIGS. 5 and 6), e.g., depending upon the thickness and/or compressibility of the tissue disposed between jaw members 72, 74, to grasp tissue between jaw members 72, 74 and apply a closure force to the grasped tissue. As an alternative to distal movement of drive shaft 88 approximating jaw members 72, 74, it is also contemplated that the opposite configuration be provided, e.g., wherein proximal movement of drive shaft 88 approximates jaw members 72, 74. Reference to a cam pin, as utilized herein, includes both continuous pins as well as split pins, e.g., wherein first and second pin portions cooperate to define the cam pin.

Upon reaching a threshold closure force applied to tissue grasped between first and second jaw members 72, 74, further movement of carriage 84 distally against compression spring 86, e.g., in response to further actuation of movable handle 34 towards fixed handle 32, compresses compression spring 86 to reduce the translation of compression spring 86 or inhibit further translation of compression spring 86 distally (due to the resistive force applied by tissue inhibiting further closure of jaw members 72, 74) such that corresponding translation of drive shaft 88 and, thus, pivoting of first and second jaw members 72, 74 to grasp tissue therebetween, is reduced or eliminated. In this manner, the closure force applied to tissue grasped between jaw members 72, 74 is regulated to maintain a closure force or closure force within a closure force range. In aspects, compression spring 86 is retained in a pre-compressed position within drive assembly 80; thus, the compression of compression spring 86 as detailed herein is relative to the initial condition of compression spring 86 within drive assembly 80 (which may be a pre-compressed initial condition or an uncompressed initial condition).

In aspects, the closure force applied to the grasped tissue in the approximated position of jaw members 72, 74 may be regulated such that the closure pressure, measured at a jaw centroid along the lengths of jaw members 72, 74, may be in a range of (or the jaw pressure range may be) from about 70 psi to about 130 psi; in other aspects from about 80 psi to about 120 psi; and, in still other aspects, from about 90 psi to about 110 psi.

Continuing with reference to FIGS. 1-4, movable handle 34 includes a flange 36 extending proximally from a lower end portion 34b of movable handle 34. Flange 36 is configured to extend through an aperture 31 defined within fixed handle 32 and ultimately engage a latch 38 within fixed handle 32 that is configured to selectively lock and unlock the fixed and movable handles 32, 34 relative to one another upon sufficient actuation of movable handle 34. Upon initial movement of flange 36 through aperture 31 to engage latch 38, in response to an initial actuation of movable handle 34 towards fixed handle 32, fixed and movable handles 32, 34 are locked relative to one another to thereby latch first and second jaw members 72, 74 in an approximated position. Upon subsequent movement of flange 36 within aperture 31, in response to a subsequent actuation of movable handle 34 towards fixed handle 32, flange 36 is disengaged from latch 38 such that fixed and movable handles 32, 34 are unlocked permitting return of movable handle 34 towards its initial position and return of jaw members 72, 74 towards the spaced apart position. In aspects, flange 36 and latch 38 are omitted and movable handle 34 is manually maintained in approximation with fixed handle 32 to thereby maintain first and second jaw members 72, 74 in an approximated position.

Lower end portion 34b of movable handle 34 further includes a grasping portion 37 configured to be grasped and manipulated by a user. Grasping portion 37 includes a finger loop 39. Finger loop 39 may be closed (as shown) or may be an open loop such as, for example, defining a shepherd's hook configuration. In either configuration, the interior of finger loop 39 is configured to receive a user's fingers to enable grasping and manipulation of movable handle 34 relative to fixed handle 32, which is configured to be grasped by the user's palm and thumb.

Rotating assembly 40 includes a rotation wheel 42 engaged with outer shaft 12 within housing 20 and extending outwardly from either side of housing 20 to enable a user to manually control the orientation of outer shaft 12 and thus, end effector assembly 70, relative to housing 20, e.g., by manipulating rotation wheel 42. In aspects, rotating assembly 40 is infinitely rotatable in either direction about the longitudinal axis “A-A” to similarly rotate end effector assembly 70 relative to housing 20. Alternatively, rotating assembly 40 may have a defined range of motion. In aspects, rotating assembly 40 is positioned in close proximity to trigger assembly 50 to facilitate a user manipulating rotation wheel 42 of rotating assembly 40 with a finger, e.g., enabling the user to move their trigger finger from of trigger 52 (or finger loop 39 of movable handle 34) to rotation wheel 42 to manipulate rotation wheel 42 to rotate rotating assembly 40.

Activation assembly 60 is configured to signal generator 18 to initiate the supply of electrosurgical energy to first and second jaw members 72, 74 for sealing tissue. Activation assembly 60 includes an activation button 62 supported by a body 22 of housing 20. Activation button 62 is movable between an unactuated position and an actuated position to thereby transition an underlying electrical switch 64 between a first state and a second state. Electrical switch 64, in turn, is adapted to electrically connect to generator 18, e.g., via one or more electrical lead wires extending from electrical switch 64 through housing 20 and electrosurgical cable 14 to plug 16 to enable communication of the state of electrical switch 64 to generator 18. Generator 18, more specifically, may be configured to read an output of a corresponding pin of plug 16, e.g., the presence of a resistance, voltage, current, etc. and/or a value of the resistance, voltage, current, etc., to detect the state of electrical switch 64 and, thus, to detect whether the user has activated activation button 62. For example, generator 18 may read the first state of electrical switch 64 as corresponding to an unactivated state and the second state of electrical switch 64 as corresponding to an activated state.

In aspects where a knife is provided, trigger assembly 50 mechanically drives the knife relative to jaw members 72, 74 to cut tissue grasped between jaw members 72, 74, e.g., once the grasped tissue is sealed. The knife may be configured for mechanically cutting tissue upon deployment of the knife between jaw members 72, 74 or for electromechanically cutting tissue wherein the knife is energized and deployed between jaw members 72, 74 to cut tissue grasped therebetween. Alternatively, a static cutting element (mechanical or energizable) may be substantially fixed relative to either or both jaw members 72, 74 for cutting tissue grasped therebetween upon energization of the cutting element and/or movement of jaw members 72, 74. In any of the energizable configurations, trigger assembly 50 may include an underlying electrical switch (not shown) configured to communicate with generator 18 (FIG. 1) to signal generator 18 (FIG. 1) to initiate the supply of energy to the energizable cutting element (and, in aspects, either or both of jaw members 72, 74) to cut tissue in response to actuation of trigger assembly 50.

Turning to FIGS. 2-6, end effector assembly 70 is described as a unilateral assembly, e.g., wherein second jaw member 74 is fixed relative to outer shaft 12 and first jaw member 72 is pivotable relative to second jaw member 74 and outer shaft 12; however, a bilateral assembly, e.g., wherein both first and second jaw members 72, 74 are pivotable relative to one another and outer shaft 12, is also contemplated. Further, in order to drive relative movement of cam pin 76 through cam slots 77, 79, drive shaft 88 may be translationally fixed to cam pin 76 and outer shaft 12 translationally fixed to pivot pin 78 such that translation of drive shaft 88 moves cam pin 76 relative to jaw members 72, 74 (and, thus, cam slots 77, 79) to thereby drive relative movement of cam pin 76 through cam slots 77, 79 to pivot first jaw member 72 towards second jaw member 74. Alternatively, drive shaft 88 may be translationally fixed to pivot pin 78 and outer shaft 12 translationally fixed to cam pin 76 such that translation of drive shaft 88 moves jaw members 72, 74 (and, thus, cam slots 77, 79) relative to cam pin 76 to thereby drive relative movement of cam pin 76 through cam slots 77, 79 to pivot first jaw member 72 towards second jaw member 74. In either configuration, or any other suitable configuration, as jaw member 72 pivots relative to jaw member 74, the jaw angle “a” (FIG. 5) defined between tissue contacting surface 73, 75 of jaw members 72, 74, respectively, is varied.

Each jaw member 72, 74 of end effector assembly 70 includes an electrically conductive tissue contacting surface 73, 75, respectively. Jaw members 72, 74 are configured to grasp tissue between electrically conductive tissue contacting surfaces 73, 75 in the approximated position(s) thereof. Electrically conductive tissue contacting surfaces 73, 75 are adapted to connect to generator 18 (FIG. 1), e.g., via suitable electrical lead wires, electrically conductive structures, or combinations thereof extending through outer shaft 12, housing 20, and electrosurgical cable 14 to plug 16 (FIG. 1), to enable energization of electrically conductive tissue contacting surfaces 73, 75 with electrosurgical, e.g., Radio Frequency (RF), energy at different potentials for conducting electrosurgical energy between electrically conductive tissue contacting surface 73, 75 and through tissue grasped therebetween to seal the tissue.

Either or both jaw members 72, 74 may further include one or more stop members 71 disposed on or otherwise associated with either or both tissue contacting surfaces 73, 75 to maintain a minimum gap distance between tissue contacting surfaces 73, 75 (or to maintain a gap distance within a gap distance range between tissue contacting surfaces 73, 75) when jaw members 72, 74 are disposed in a fully approximated position, thus inhibiting electrical shorting. Stop member(s) 71 may be insulative, partly insulative, and/or electrically isolated from either or both tissue contacting surfaces 73, 75. In aspects, the minimum gap distance or the gap distance range may be from about 0.001 inches to about 0.010 inches; in other aspects from about 0.001 inches to about 0.008 inches; and, in still other aspects from about 0.001 inches to about 0.006 inches. Other suitable gap distances and ranges are also contemplated. The gap distance may be determined as the maximum gap distance between the tissue contacting surfaces 73, 75.

Continuing with reference to FIGS. 4-6, jaw member 72 may include a single jaw flag 92 defining cam slot 77 configured to receive cam pin 76 or may include a pair of spaced apart jaw flags 92 defining aligned cam slots 77 configured to receive cam pin 76. Likewise, jaw member 74 may include a single jaw flag 94 defining cam slot 79 configured to receive cam pin 76 or may include a pair of spaced apart jaw flags 94 defining aligned cam slots 79 configured to receive cam pin 76. Where both jaw members 72, 74 include a pair of spaced apart jaw flags 92, 94, the pairs of jaw flags 92, 94 may be positioned in overlapping offset relation, e.g., wherein one jaw flag 92, 94 of each pair is disposed between the jaw flags 92, 94 of the other pair or may be disposed in a nested arrangement, e.g., wherein both jaw flags 92, 94 of one pair are disposed between the jaw flags 92, 94 of the other pair. Where one jaw member 72, 74 include a pair of spaced apart jaw flags 92, 94 and the other jaw member 72, 74 includes a single jaw flag 92, 94, the single jaw flag 92, 94 may be disposed between the pair of jaw flags 92, 94 of the other jaw member 72, 74 or may be disposed exteriorly of both jaw flags 92, 94 of the other jaw member 72, 74. Where both jaw members 72, 74 include a single jaw flag 92, 94, the jaw flags 92, 94 are disposed in side-by-side relation relative to one another (with or without intervening structure(s) disposed therebetween). For simplicity, and without limitation, reference herein is made to a single jaw flag 92, 94 of each jaw member 72, 74, keeping in mind that the aspects and features detailed herein may likewise apply to dual flag configurations for either or both jaw members 72, 74.

In unilateral configurations (such as the configuration shown in FIG. 4-6), cam slot 79 of fixed second jaw member 74 extends linearly in substantially parallel or coaxial orientation relative to longitudinal axis “A-A” (FIG. 1) and functions to guide translation of cam pin 76. That is, since fixed second jaw member 74 is stationary, cam pin 76 need not exert force to affect motion of fixed second jaw member 74. Thus, cam slot 79 may be provided for guiding purposes or, in other unilateral configurations, cam slot 79 of fixed second jaw member 74 (and, in aspects a portion or the entirety of the jaw flag(s) 94 thereof) may be omitted.

However, as noted above, the present disclosure also contemplates bilateral configurations. Thus, although the aspects and features of the present disclosure are detailed below with respect to cam slot 77 of movable first jaw member 72 in a unilateral configuration, the aspects and features of the present disclosure are equally applicable for use in a bilateral configuration wherein second jaw member 74 is also movable and cam slot 79 of second jaw member 74 is configured in the same manner as cam slot 77 of first jaw member 72 except that cam slot 79 of second jaw member 74 is inverted relative to cam slot 77 of first jaw member 72. Accordingly, both jaw members 72, 74 are moved equally (in amount of travel and force exerted) and oppositely between the spaced apart and approximated positions.

Regardless of whether a unilateral or bilateral configuration is provided, cam pin 76 is constrained to substantially longitudinal translation, e.g., along axis “A-A” (FIG. 1). This constraint on cam pin 76 may be provided by drive shaft 88 (FIGS. 2 and 3, e.g., via the engagement of cam pin 76 with drive shaft 88), may be provided by cam slot 79 (as noted above), and/or may be provided by any other suitable structures associated with shaft 12 and/or end effector assembly 70 (see FIG. 1).

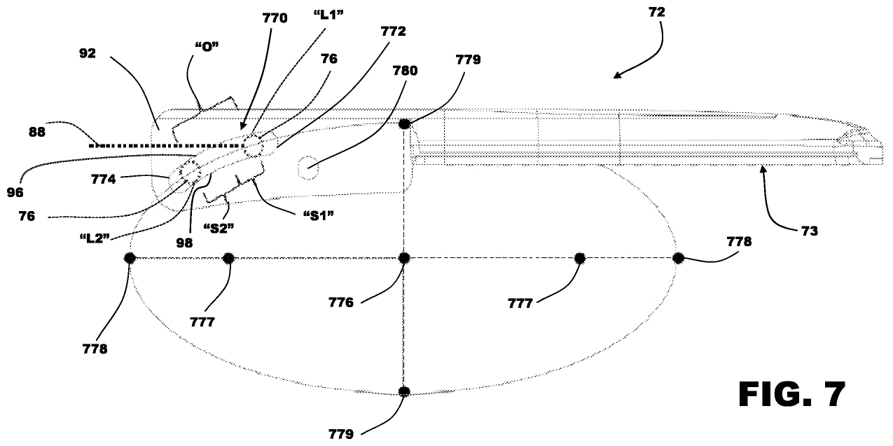

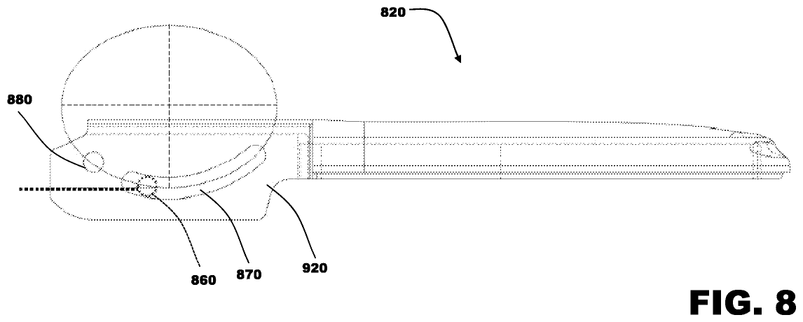

The orientation and/or positioning of cam slot 77 within jaw flag 92 of jaw member 72 may vary depending upon, for example, the movable component(s) that approximate jaw members 72, 74; the direction of relative movement of the movable component(s) that approximate jaw members 72, 74; and/or the position of cam slot 77 relative to pivot pin 78. More specifically, as noted above, and with momentary additional reference to FIGS. 2 and 3, translation of drive shaft 88 may move cam pin 76 through cam slot 77 or, alternatively, translation of drive shaft 88 may move cam slot 77 about cam pin 76. Likewise, jaw member 72 may be configured to pivot towards an approximated position (FIGS. 5 and 6) in response to distal movement of cam pin 76 relative to cam slot 77 or, alternatively, jaw member 72 may be configured to pivot towards an approximated position (FIGS. 5 and 6) in response to proximal movement of cam pin 76 relative to cam slot 77. Additionally, cam slot 77 may be positioned proximally of pivot pin 78 or, alternatively, may be positioned distally of pivot pin 78. As can be appreciated, the orientation and/or position of cam slot 77 may vary depending upon one or more of the above configurations. Thus, although two exemplary positions and orientations of cam slots are detailed below and illustrated in FIGS. 7 and 8, the present disclosure is not limited thereto; rather, any suitable orientations and/or positions of cam slots may be provided.

Referring still to FIGS. 4-6, the configuration of cam slot 77 impacts the jaw force applied to tissue disposed between jaw members 72, 74 at any given position of cam pin 76 along the actuation travel path of cam pin 76 through cam slot 77. Likewise, the jaw force profile, defined as the change in jaw force applied to tissue disposed between jaw members 72, 74 as the angular position (e.g., jaw angle “a” (FIG. 5)) of jaw member 72 relative to jaw member 74 is changed in response to movement of cam pin 76 along the actuation travel path of cam pin 76 through cam slot 77, depends at least in part upon the configuration of cam slot 77. Exemplary cam slot configurations for use with jaw member 72 and/or any other suitable jaw member(s) are detailed below and illustrated in FIGS. 7 and 8.

Turning to FIG. 7, a cam slot 770 is shown defined through jaw flag 92 of jaw member 72. Cam slot 770 is positioned proximally of the pivot location (e.g., defined by pivot aperture 780) and is configured such that proximal movement of cam pin 76 through cam slot 770 moves jaw member 72 from the spaced apart position (e.g., corresponding to location “L1” of cam pin 76) to an approximated position (e.g., corresponding to location “L2” of cam pin 76). However, as noted above, other orientations and/or positions are also contemplated. Further, although detailed with respect to jaw member 72 of electrosurgical forceps 10 (FIG. 1), cam slot 770 may additionally or alternatively be utilized in any other suitable surgical instrument.

In aspects, stop features (e.g., mechanical stops associated with movable handle 34 and/or drive assembly 80 (see FIG. 2) and/or software stops (such as, for example, in powered or robotic implementations)) defining a range of motion of drive assembly 80 (FIG. 2)) limit the actuation travel path of cam pin 76 through cam slot 770 to less than the full extent of cam slot 770. That is, rather than cam pin 76 bottoming out at distal and/or proximal ends 772, 774 of cam slot 770, the stop feature(s) may establish the distal-most position of cam pin 76 (corresponding to location “L1” and the fully spaced apart position of jaw members 72, 74 (FIG. 4)) proximally spaced from distal end 772 of cam slot 770 and/or the proximal-most position of cam pin 76 (corresponding to location “L2” and the fully approximated position of jaw members 72, 74 (FIG. 6)) distally spaced from proximal end 774 of cam slot 770. Thus, in aspects, the operative portion “O” of cam slot 770, defined as the portion of cam slot 770 through which cam pin 76 is configured to travel between the fully spaced apart and fully approximated positions, may be less than the entire extent of cam slot 770. Accordingly, since cam pin 76 is confined to the operative portion “O” of cam slot 770, the configurations of any portions of cam slot 770 outside of the operative portion “O,” e.g., portions of cam slot 770 between location “L1” and distal end 772 of cam slot 770 and/or between location “L2” and proximal end 774 of cam slot 770, do not impact the jaw force applied to tissue disposed between jaw members 72, 74 and, thus, need not be configured to achieve a desired jaw force or jaw force profile. For example, the portion(s) of cam slot 770 outside of operative portion “O” may be configured for another purpose, e.g., to facilitate manufacturing or assembly, etc., and/or may be provided to maintain clearance and/or enable overtravel. In aspects, the portion(s) of cam slot 770 outside of operative portion “O” may define different configurations, e.g., radiused arcs, other arc curvatures, linear configurations, etc.

Continuing with reference to FIG. 7, and with additional reference to FIGS. 2 and 4, operative portion “O” of cam slot 770, in aspects, can be split into two distinct sections: a first, substantially spring-independent travel section “S1,” wherein compression spring 86 is substantially maintained in its initial condition while being translated to translate drive shaft 88 to thereby move cam pin 76 through spring-independent travel section “S1” of cam slot 770, and a second, spring-influenced travel section “S2,” wherein compression spring 86 is compressed from its initial condition while being translated (or to inhibit further translation) to thereby regulate the translation of drive shaft 88 and, thus, to regulate movement of cam pin 76 through spring-influenced travel section “S2” of cam slot 770. It is noted that the transition between sections “S1” and “S2” is not necessarily defined by a physical change in cam slot 770 or a fixed location along cam slot 770 but, rather, is defined as the location of cam pin 76 along cam slot 770 wherein compression spring 86 is compressed from its initial condition. As the location of cam pin 76 along cam slot 770 wherein compression spring 86 is compressed from its initial condition varies depending upon the size and/or location of tissue between jaw members 72, 74, the proportions of operative portion “O” defined by sections “S1” and “S2” are not fixed or pre-determined. That is, while sections “S1” and “S2” together define operative portion “O” of cam slot 770, the proportions of operative portion “O” defined by sections “S1” and “S2” vary depending upon the size and/or location of tissue between jaw members 72, 74. More specifically, as the size of tissue disposed between jaw members 72, 74 decreases and/or as tissue is more distally-positioned between jaw members 72, 74, section “S1” defines an increasingly greater proportion of operative portion “O” of cam slot 770 (and, thus, section “S2” defines a decreasingly smaller proportion of operative portion “O” of cam slot 770) due to the fact that jaw members 72, 74 are required to be approximated further towards the approximated position before contacting the tissue and engaging compression spring 86. In aspects where tissue is very small and/or distally positioned, section “S1” is maximized and section “S2” is minimized. On the other hand, as the size of tissue disposed between jaw members 72, 74 increases and/or as tissue is more proximally-positioned between jaw members 72, 74, section “S1” defines a decreasingly smaller proportion of operative portion “O” of cam slot 770 (and, thus, section “S2” defines an increasingly greater proportion of operative portion “O” of cam slot 770) due to the fact that jaw members 72, 74 are not required to be approximated very far to contact the tissue and engaging compression spring 86. In aspects where tissue is very large and/or proximally positioned, section “S1” is minimized and section “S2” is maximized.

At least a portion of operative portion “O” of cam slot 770 defines an elliptical arc. More specifically, operative portion “O” of cam slot 770 may define a continuous elliptical arc; may define different elliptical arcs (having different eccentricities, different centers, different vertices, different co-vertices, and/or different foci); or may include a first portion defining an elliptical arc while another portion defines a different configuration, e.g., a radiused arc, another arc curvature, a linear configuration, etc.

The elliptical arc of operative portion “O” of cam slot 770 is defined by a center 776, a pair of foci 777, a pair of vertices 778, and a pair of co-vertices 779, according to the equation:

c²=a²−b² Equation (1)

where c is the absolute value of the distance between center 776 and a focus 777 (the distance between center 776 and each focus 777 is the same), a is the absolute value of the distance between center 776 and a vertex 778 along the major axis (the distance between center 776 and each vertex 778 is the same), and b is the absolute value of the distance between center 776 and a co-vertex 779 along the minor axis (the distance between center 776 and each co-vertex 779 is the same). The eccentricity, E, of the ellipse is defined according to the equation:

$$E = \frac{c}{a} \qquad Equation ( 2 )$$

In aspects, the eccentricity, E of the ellipse is from about 0.65 to about 0.95; in other aspects, from about 0.68 to about 0.92; or, in still other aspects, from about 0.70 to about 0.90. The eccentricity, E, of cam slot 770 illustrated in FIG. 7 is, for example and without limitation, about 0.895.

In aspects, the elliptical arc of operative portion “O” of cam slot 770, may be defined between a vertex 778 and a co-vertex 779 without intersecting either, such that the elliptical arc is contained within one quadrant of an ellipse. In other aspects, the elliptical arc of operative portion “O” of cam slot 770 may intersect a vertex 778 or a co-vertex 779 such that the elliptical arc extends at least partially within two adjacent quadrants of an ellipse. In aspects, the elliptical arc of operative portion “O” may define a major axis (between the vertices 778) that is substantially parallel relative to longitudinal axis “A-A” of electrosurgical forceps 10 (FIG. 1) and, thus, a minor axis (between the co-vertices 779) that is substantially perpendicular relative to longitudinal axis “A-A” of electrosurgical forceps 10 (FIG. 1). Alternatively, the elliptical arc of operative portion “O” of cam slot 770 may be oriented such that the major axis defines a smaller (non-zero, absolute value) angle relative to the longitudinal axis “A-A” of electrosurgical forceps 10 (FIG. 1) as compared to the minor axis. Other orientations are also contemplated.

Due to manufacturing considerations, design considerations, tolerances, and/or for other reasons, the elliptical arc of operative portion “O” of cam slot 770, may not be perfectly elliptical. Rather, the elliptical arc may approximate a perfectly elliptical configuration such that any point along the elliptical arc deviates from a perfect ellipse (in either direction normal to the perfect ellipse) a distance of, in aspects, less than or equal to about +0.006 inches; in other aspects, less than or equal to about +0.004 inches; or in still other aspects, less than or equal to about #0.002 inches.

The elliptical arc of operative portion “O” of cam slot 770, in aspects, may be defined through a center of cam slot 770, along an upper edge 96 of jaw flag 92 that defines cam slot 770, and/or along a lower edge 98 of jaw flag 92 that defines cam slot 770. In aspects, at least upper or lower edge 96, 98 may define the elliptical arc when that edge 96, 98 functions as the load bearing edge during movement of jaw member 72 towards an approximated position. For example, in the configuration illustrated in FIG. 7, upper edge 96 is the load bearing edge against which cam pin 76 is urged during movement of cam pin 76 from location “L1” towards location “L2” to move jaw member 72 towards an approximated position (e.g., whereby cam pin 76 urges jaw member 72 to rotate clockwise in the orientation illustrated in FIG. 7 as cam pin 76 is moved from location “L1” towards location “L2”). However, other configurations are also contemplated.

Referring still to FIG. 7, the angle between the force vector applied by cam pin 76 to jaw flag 92 (e.g., in response to urging cam pin 76 through cam slot 770) and the load bearing edge 96, 98 of cam slot 770 at the location of cam pin 76 (referred to herein as the “force vector angle”) determines the force that is imparted from cam pin 76 to pivot jaw flag 92 at each location of cam pin 76. This force, in turn, is related to the jaw force applied by jaw member 72 to tissue disposed between jaw members 72, 74 (see FIGS. 5 and 6). Thus, at least a portion of cam slot 770 can be configured such that an appropriate force vector angle is established at each location of cam pin 76 along the at least a portion of cam slot 770 to thereby achieve a substantially constant force applied from jaw member 72 to tissue at each of the locations of cam pin 76 and, thus, regardless of the jaw angle “a” (see FIG. 5). Accordingly, larger and/or less compressible tissues (requiring a relatively larger jaw angles “a” (see FIG. 5)) can be effectively sealed, as can smaller and/or more compressible tissues (requiring relatively smaller jaw angles “a” (see FIG. 5)).

It has been found that the elliptical arcs provided in accordance with the present disclosure, e.g., the elliptical arc of operative portion “O” of cam slot 770, provide the appropriate force vector angles at each location of cam pin 76 to achieve a substantially constant force applied from jaw member 72 to tissue regardless of the position of cam pin 76 along at least a section thereof, e.g., at least a section of operative portion “O” of cam slot 770 and, thus, regardless of the corresponding jaw angle “a” (see FIG. 5). In aspects, the substantially constant force is applied along an entirety of the operative portion “O” of cam slot 770. In aspects, the substantially constant force is applied along spring-independent travel section “S1” of operative portion “O” of cam slot 770. In additional or alternative aspects, the substantially constant force is applied along spring-influenced travel section “S2” of operative portion “O” of cam slot 770. It is noted that, although compression spring 86 (FIG. 2) regulates the force applied in spring-influenced travel section “S2,” compression spring 86, drive assembly 80, and end effector assembly 70 (see FIGS. 2 and 4) may be configured such that the force applied by compression spring 86 is substantially linear relative to an amount of compression thereof (e.g., according to F=kx), thus facilitating providing the substantially constant force within spring-influenced travel section “S2” of operative portion “O” of cam slot 770.

In addition to the configuration of cam slot 770 (e.g., wherein at least a portion of operative portion “O” of cam slot 770 defines an elliptical arc) enabling substantially constant jaw force, the configuration of cam slot 770 also impacts the angular rate (e.g., change in jaw angle “a” (FIG. 5)) of pivoting of jaw member 72 relative to jaw member 74 in response to a given translation distance of cam pin 76 along the actuation travel path of cam pin 76 through cam slot 770 depends at least in part upon the configuration of cam slot 770. More specifically, the above detailed elliptical arc of operative portion “O” of cam slot 770 may provide a variable closure rate wherein, in response to the same translation distance of cam pin 76 through first and second different portions of cam slot 770, jaw member 72 is approximated a different amount (e.g., resulting in a different change of jaw angle “a” (FIG. 5)) as cam pin 76 is translated through the first portion of cam slot 770 compared to the second portion of cam slot 770.

Turning to FIG. 8, another cam slot 870 is shown defined through a jaw flag 920 of another jaw member 820. Jaw member 820 may be similar to and include any of the features of jaw member 72 (FIGS. 4-6) and may be configured for use with electrosurgical forceps 10 (FIG. 1) or any other suitable instrument. Cam slot 870 is positioned distally of the pivot location (e.g., defined by pivot aperture 880) and is configured such that distal movement of the cam pin 860 through cam slot 870 moves jaw member 820 from the spaced apart position to an approximated position. Although disposed in a different orientation and position compared to cam slot 770 (FIG. 7), cam slot 870 may otherwise include any of the aspects and features of cam slot 770 (FIG. 7), detailed above. The eccentricity, E, of cam slot 870 illustrated in FIG. 8 is, for example and without limitation, about 0.706.

While aspects and features of the present disclosure are illustrated and described above as being utilized with a handheld electrosurgical forceps (whether motorized or manually powered), the aspects and features of the present disclosure be likewise apply to surgical instruments for robotic surgical systems. Such systems employ various robotic elements to assist the user and allow remote operation (or partial remote operation) of surgical instrumentation. Various robotic arms, gears, cams, pulleys, electric and mechanical motors, etc. may be employed for this purpose and may be designed with a robotic surgical system to assist the user during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to an operating theater or located in a remote location. In this instance, one team of users may prep a patient for surgery and configure the robotic surgical system with one or more of the instruments disclosed herein while another user (or group of user) remotely control the instruments via the robotic surgical system. As can be appreciated, a highly skilled user may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the user to produce a corresponding movement of the working ends of any type of surgical instrument (e.g., end effectors, graspers, knifes, scissors, etc.) which may complement the use of one or more of the aspects described herein. The movement of the master handles may be scaled so that the working ends have a corresponding movement that is different, smaller or larger, than the movement performed by the operating hands of the user. The scale factor or gearing ratio may be adjustable so that the operator can control the resolution of the working ends of the surgical instrument(s).

The master handles may include various sensors to provide feedback to the user relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the user with enhanced tactile feedback simulating actual operating conditions. The master handles may also include a variety of different actuators for delicate tissue manipulation or treatment further enhancing the user's ability to mimic actual operating conditions.

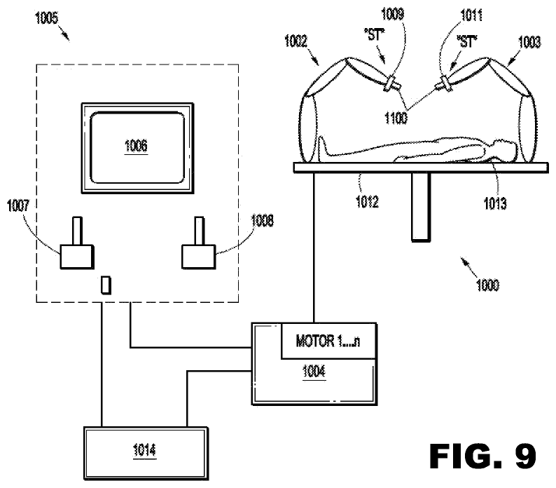

Referring to FIG. 9, a medical workstation of a robotic surgical system such as detailed above is shown generally as workstation 1000 and generally includes a plurality of robot arms 1002, 1003; a control device 1004; and an operating console 1005 coupled with the control device 1004. The operating console 1005 may include a display device 1006, which may be set up in particular to display three-dimensional images; and manual input devices 1007, 1008, by means of which a user may be able to telemanipulate the robot arms 1002, 1003 in a first operating mode.

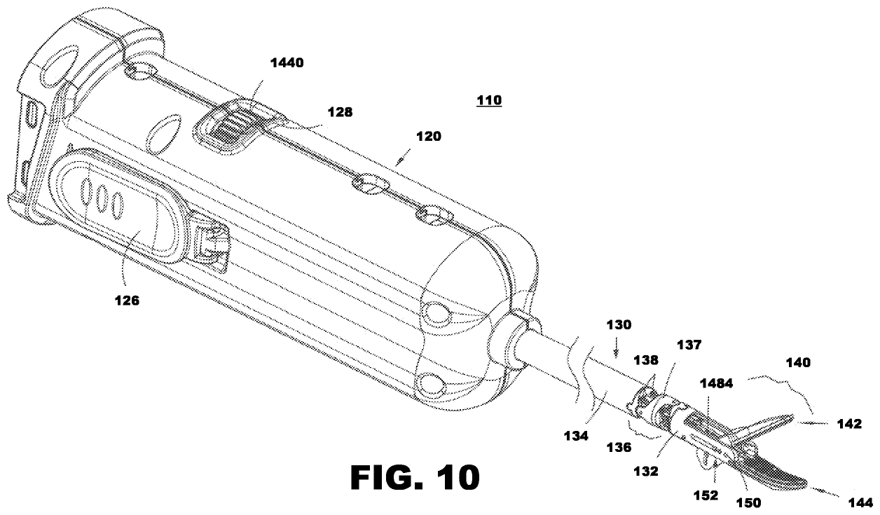

Each of the robot arms 1002, 1003 may include a plurality of members, which are connected through joints, and an attaching device 1009, 1011, to which may be attached, for example, a surgical tool “ST” supporting an end effector 1100. The surgical tool “ST” may include, for example, robotic electrosurgical forceps 110 (FIG. 10), as detailed below.

The robot arms 1002, 1003 may be driven by electric drives (not shown) that are connected to the control device 1004. The control device 1004 (e.g., a computer) may be set up to activate the drives, in particular by means of a computer program, in such a way that the robot arms 1002, 1003, their attaching devices 1009, 1011 and thus the surgical tool “ST” (e.g., robotic electrosurgical forceps 110 (FIG. 10)) execute a desired movement according to a movement defined by means of the manual input devices 1007, 1008. The control device 1004 may also be set up in such a way that it regulates the movement of the robot arms 1002, 1003 and/or of the drives.

The medical workstation 1000 may be configured for use on a patient 1013 lying on a patient table 1012 to be treated in a minimally invasive manner by means of the end effector 1100. The medical workstation 1000 may also include more than two robot arms 1002, 1003, the additional robot arms likewise being connected to the control device 1004 and being telemanipulatable by means of the operating console 1005. A medical instrument or surgical tool (including an end effector 1100) may also be attached to the additional robot arm. Medical workstation 1000 may include a database 1014, in particular coupled to the control device 1004, in which are stored, for example, pre-operative data from the patient/living being 1013 and/or anatomical atlases.

Turning to FIG. 10, a robotic electrosurgical forceps 110 provided in accordance with the present disclosure and configured, for example, for use with the robotic surgical system of FIG. 9, generally includes a housing 120, a shaft 130 extending distally from housing 120, an end effector assembly 140 extending distally from shaft 130, and an actuation assembly (not shown) disposed within housing 120 and operably associated with end effector assembly 140.

Housing 120 of instrument 110 encloses the actuation assembly therein and includes through holes through which input couplers of the actuation assembly extend to enable a robotic arm to couple to actuation assembly and selectively actuate the various features thereof. A pair of latch levers 126 (only one of which is illustrated in FIG. 10) extending outwardly from opposing sides of housing 120 enable releasable engagement of housing 120 with the robotic arm. A window 128 defined through housing 120 permits thumbwheel 1440 to extend therethrough to enable manual manipulation of thumbwheel 1440 from the exterior of housing 120 to permit manual opening and closing of end effector assembly 140.

Shaft 130 of instrument 110 includes a distal clevis segment 132, a proximal segment 134, and an articulating section 136 disposed between the distal clevis and proximal segments 132, 134, respectively. Articulating section 136 includes one or more articulating components 137, e.g., links, joints, etc. A plurality of articulation cables 138, e.g., four (4) articulation cables, or other suitable actuators, extend through articulating section 136. More specifically, articulation cables 138 are operably coupled to distal clevis segment 132 of shaft 130 at the distal ends thereof and extend proximally from distal clevis segment 132 of shaft 130, through articulating section 136 of shaft 130 and proximal segment 134 of shaft 130, and into housing 120, wherein articulation cables 138 operably couple with an articulation subassembly of the actuation assembly to enable selective articulation of distal clevis segment 132 (and, thus end effector assembly 140) relative to proximal segment 134 and housing 120, e.g., about at least two axes of articulation (yaw and pitch articulation, for example).

End effector assembly 140 includes first and second jaw members 142, 144, respectively, pivotably connected about a pivot pin 150. Jaw members 142, 144 may be configured similar to and incorporate any of the features of jaw members 72, 74 (FIGS. 4-6), as detailed above.

A drive shaft, also referred to as a drive rod 1484 is operably coupled to cam slot assembly 152 of end effector assembly 140, e.g., engaged with the cam pin thereof, such that longitudinal actuation of drive rod 1484 pivots jaw member 142 relative to jaw member 144 between the spaced apart and approximated positions. More specifically, urging drive rod 1484 proximally pivots jaw member 142 relative to jaw member 144 towards the approximated position while urging drive rod 1484 distally pivots jaw member 142 relative to jaw member 144 towards the spaced apart position. However, other suitable mechanisms and/or configurations for pivoting jaw member 142 relative to jaw member 144 between the spaced apart and approximated positions in response to selective actuation of drive rod 1484 are also contemplated. Drive rod 1484 extends proximally from end effector assembly 140 through shaft 130 and into housing 120 wherein drive rod 1484 is operably coupled with a jaw drive subassembly of the actuation assembly to enable selective actuation of end effector assembly 140 to grasp tissue therebetween and apply a jaw force within an appropriate jaw force range. The jaw drive subassembly may be configured similar to drive assembly 80 (FIG. 2) detailed above except that suitable gearing, pulleys, linkages, and/or other components are utilized to move the compression spring 86 (FIG. 2) and, thus, drive rod 1484, in response to a rotational input from a robotic arm rather than manual actuation of movable handle 34 (FIG. 1). Cam slot assembly 152 may include one or more cam slots defined within one or both of jaw members 72, 74 that are configured to receive the cam pin of cam slot assembly 152. The cam slots may be configured in accordance with any of the aspects and features of cam slots detailed herein.

The actuation assembly of instrument 110, as noted above, is configured to operably interface with a surgical robotic system when instrument 110 is mounted on a robotic arm thereof, to enable robotic operation of the actuation assembly to provide the above detailed functionality. That is, the surgical robotic system selectively provides inputs, e.g., rotational inputs to the input couplers of the actuation assembly to articulate end effector assembly 140, grasp tissue between jaw members 142, 144, and/or cut tissue grasped between jaw members 142, 144.

Aspects of this disclosure may be further described by reference to the following numbered paragraphs:

1. A surgical instrument (10, 110), comprising: a pivot pin (78, 150); a cam pin (76); and first and second jaw members (72, 74; 142, 144) pivotably coupled to one another about the pivot pin (78, 150), the first jaw member (72, 142) defining a cam slot (770, 870) having the cam pin (76, 860) received therein such that movement of the cam pin (76, 860) through the cam slot (770, 870) pivots the first jaw member (72, 142) relative to the second jaw member (74, 144) from a spaced apart position towards an approximated position to grasp tissue between the first and second jaw members (72, 74; 142, 144), wherein at least a portion of the cam slot (770, 870) defines an elliptical arc to facilitate application of a substantially constant jaw force from the first jaw member (72, 142) to tissue grasped between the first and second jaw members (72, 74; 142, 144) when the cam pin (76, 860) is disposed at any position within the portion of the cam slot (770, 870).

2. The surgical instrument (10, 110) according to paragraph 1, wherein the elliptical arc defines an eccentricity of from about 0.70 to about 0.90.

3. The surgical instrument (10, 110) according to paragraph 1 or 2, wherein the elliptical arc deviates from a perfect ellipse by an amount less than or equal to about 0.006 inches at any point along the elliptical arc in either direction normal to the perfect ellipse.

4. The surgical instrument (10, 110) according to paragraph 1 or 2, wherein the elliptical arc deviates from a perfect ellipse by an amount less than or equal to about 0.004 inches at any point along the elliptical arc in either direction normal to the perfect ellipse.

5. The surgical instrument (10, 110) according to any one of paragraphs 1-4, wherein the elliptical arc is disposed between a vertex (778) and a co-vertex (779) of an ellipse defining the elliptical arc.

6. The surgical instrument (10, 110) according to any one of paragraphs 1-4, wherein the elliptical arc intersects a vertex (778) or a co-vertex (779) of an ellipse defining the elliptical arc.

7. The surgical instrument (10, 110) according to any preceding paragraph, wherein the portion of the cam slot (770, 870) is an operative portion “O” of the cam slot (770, 870) defined between a proximal-most position of the cam pin (76, 860) within the cam slot (770, 870) and a distal-most position of the cam pin (76, 860) within the cam slot (770, 870).

8. The surgical instrument (10, 110) according to paragraph 7, wherein at least one of: the proximal-most position is distally spaced from a proximal end (774) of the cam slot (770, 870) or the distal-most position is proximally spaced from a distal end (772) of the cam slot (770, 870), such that the portion of the cam slot (770, 870) is less than an entire extent of the cam slot (770, 870).

9. The surgical instrument (10, 110) according to any preceding paragraph, further comprising a drive assembly (80), including: a compression spring (86); and a drive shaft (88, 1484) coupling the compression spring (86) with the cam pin (76, 860) such that translation of the compression spring (86) translates the drive shaft (88, 1484) to thereby move the cam pin (76, 860) through the cam slot (770, 870).

10. The surgical instrument (10, 110) according to paragraph 9, wherein the cam slot (770, 870) defines a first section (“S1”) and a second section (“S2”), wherein movement of the cam pin (76, 860) through the first section (“S1”) of the cam slot (770, 870) is substantially independent of a spring force associated with the compression spring (86) and wherein movement of the cam pin (76, 860) through the second section (“S2”) of the cam slot (770, 870) is dependent upon the spring force associated with the compression spring (86).

11. The surgical instrument (10, 110) according to paragraph 10, wherein the portion of the cam slot (770, 870) includes at least one of the first section (“S1”) or the second section (“S2”).

12. The surgical instrument (10) according to any one of paragraphs 9-11, wherein the drive assembly (80) is driven by a manually-actuated handle (34).

13. The surgical instrument (110) according to any one of paragraphs 9-11, wherein the drive assembly (80) is driven by a robotic arm (102, 103).

14. The surgical instrument (10, 110) according to any preceding paragraph, wherein the cam slot (770, 870) is configured such that movement of the cam pin (76, 860) a distance through a first section of the cam slot (770, 870) pivots the first jaw member (72, 142) relative to the second jaw member (74, 144) a first angular amount and wherein movement of the cam pin (76, 860) the distance through a second section of the cam slot (770, 870) pivots the first jaw member (72, 142) relative to the second jaw member (74, 144) a second, different angular amount.

15. The surgical instrument (10, 110) according to any preceding paragraph, wherein both the first and second jaw members (72, 74; 142, 144) are pivotable relative to one another from the spaced apart position towards the approximated position to grasp tissue between the first and second jaw members (72, 74; 142, 144).

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

What is claimed is:

1. A surgical instrument, comprising:
a pivot pin;
a cam pin; and
first and second jaw members pivotably coupled to one another about the pivot pin, the first jaw member defining a cam slot having the cam pin received therein such that movement of the cam pin through the cam slot pivots the first jaw member relative to the second jaw member from a spaced apart position towards an approximated position to grasp tissue between the first and second jaw members,
wherein at least a portion of the cam slot defines an elliptical arc to facilitate application of a substantially constant jaw force from the first jaw member to tissue grasped between the first and second jaw members when the cam pin is disposed at any position within the portion of the cam slot.

2. The surgical instrument according to claim 1, wherein the elliptical arc defines an eccentricity of from about 0.70 to about 0.90.

3. The surgical instrument according to claim 1, wherein the elliptical arc deviates from a perfect ellipse by an amount less than or equal to about 0.006 inches at any point along the elliptical arc in either direction normal to the perfect ellipse.

4. The surgical instrument according to claim 1, wherein the elliptical arc is disposed between a vertex and a co-vertex of an ellipse defining the elliptical arc.

5. The surgical instrument according to claim 1, wherein the portion of the cam slot is an operative portion of the cam slot defined between a proximal-most position of the cam pin within the cam slot and a distal-most position of the cam pin within the cam slot.

6. The surgical instrument according to claim 5, wherein at least one of: the proximal-most position is distally spaced from a proximal end of the cam slot or the distal-most position is proximally spaced from a distal end of the cam slot, such that the portion of the cam slot is less than an entire extent of the cam slot.

7. The surgical instrument according to claim 1, further comprising a drive assembly, including:
a compression spring; and
a drive shaft coupling the compression spring with the cam pin such that translation of the compression spring translates the drive shaft to thereby move the cam pin through the cam slot.

8. The surgical instrument according to claim 7, wherein the cam slot defines a first section and a second section, wherein movement of the cam pin through the first section of the cam slot is substantially independent of a spring force associated with the compression spring and wherein movement of the cam pin through the second section of the cam slot is dependent upon the spring force associated with the compression spring.

9. The surgical instrument according to claim 8, wherein the portion of the cam slot includes at least one of the first section or the second section.

10. The surgical instrument according to claim 1, wherein the cam slot is configured such that movement of the cam pin a distance through a first section of the cam slot pivots the first jaw member relative to the second jaw member a first angular amount and wherein movement of the cam pin the distance through a second section of the cam slot pivots the first jaw member relative to the second jaw member a second, different angular amount.

11. A surgical instrument, comprising:
a drive assembly including a compression spring and a drive shaft, wherein translation of the compression spring translates the drive shaft;
a cam pin, wherein the drive shaft is engaged to the cam pin such that translation of the drive shaft moves the cam pin; and
first and second jaw members pivotably coupled to one another, the first jaw member defining a cam slot having the cam pin received therein such that movement of the cam pin through the cam slot pivots the first jaw member relative to the second jaw member from a spaced apart position towards an approximated position to grasp tissue between the first and second jaw members,
wherein at least a portion of the cam slot defines an elliptical arc to facilitate application of a substantially constant jaw force from the first jaw member to tissue grasped between the first and second jaw members when the cam pin is disposed at any position within the portion of the cam slot.

12. The surgical instrument according to claim 11, wherein the elliptical arc defines an eccentricity of from about 0.70 to about 0.90.

13. The surgical instrument according to claim 11, wherein the elliptical arc deviates from a perfect ellipse by an amount less than or equal to about 0.006 inches at any point along the elliptical arc in either direction normal to the perfect ellipse.

14. The surgical instrument according to claim 11, wherein the elliptical arc is disposed between a vertex and a co-vertex of an ellipse defining the elliptical arc.

15. The surgical instrument according to claim 11, wherein the portion of the cam slot is an operative portion of the cam slot defined between a proximal-most position of the cam pin within the cam slot and a distal-most position of the cam pin within the cam slot.

16. The surgical instrument according to claim 15, wherein at least one of: the proximal-most position is distally spaced from a proximal end of the cam slot or the distal-most position is proximally spaced from a distal end of the cam slot, such that the portion of the cam slot is less than an entire extent of the cam slot.

17. The surgical instrument according to claim 11, wherein the cam slot defines a first section and a second section, wherein movement of the cam pin through the first section of the cam slot is substantially independent of a spring force associated with the compression spring and wherein movement of the cam pin through the second section of the cam slot is dependent upon the spring force associated with the compression spring.

18. The surgical instrument according to claim 17, wherein the portion of the cam slot includes at least one of the first section or the second section.

19. The surgical instrument according to claim 11, wherein the cam slot is configured such that movement of the cam pin a distance through a first section of the cam slot pivots the first jaw member relative to the second jaw member a first angular amount and wherein movement of the cam pin the distance through a second section of the cam slot pivots the first jaw member relative to the second jaw member a second, different angular amount.

20. The surgical instrument according to claim 11, wherein the drive assembly is driven by one of: a manually-actuated handle or a robotic arm.
